(19) United States
(12) Patent Application Publication
(10) Pub. No.: US 2022/0338910 A1
(43) Pub. Date: Oct. 27, 2022

(54) METHOD AND DEVICES FOR IMPLANTATION OF BIOLOGIC CONSTRUCTS

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 17/761,826

(22) PCT Filed: Sep. 24, 2020
(86) PCT No.: PCT/US2020/052491

§ 371 Date: Mar. 18, 2022

Related U.S. Application Data
(60) Provisional application No. 62/905,988, filed on Sep. 25, 2019.
(60) Provisional application No. 63/043,210, filed on Jun. 24, 2020.

(51) Int. Cl. A61B 17/80

(52) U.S. Cl. CPC A61B 17/8085

(57) ABSTRACT

Methods and apparatus for delivering a sheet-like implant to a target site including a means of deploying and orienting the sheet-like implant within the body.

### FIELD OF THE INVENTIONS

The inventions described below relate to the field of arthroscopic surgery and more specifically to implantation of biologic constructs.

### BACKGROUND OF THE INVENTIONS

Biologic constructs are a family of biologically derived implants to promote tissue growth or to patch and repair tissue defects and tears. These include the repair of arthritic cartilage, the joining of tendons to bone and the bridging of degenerated rotator cuff in the shoulder. Biologic constructs, and graft material such as platelet rich fibrin membrane, acellular dermal allograft, (MTF) and xenograft materials (Pegasus Biologics) and graft patches (Wright Medical Graftjacket) have enabled the reconstruction and treatment of previously untreatable and irreparable musculoskeletal injuries and pathologies. Biologic constructs now occupy an increasingly important place in the orthopedic surgeon's armamentarium.

One of the key problems with biologic constructs is that the delivery instrumentation has not kept pace with advances in these implants. For example, fluid seals effectively hold fluid, but do not allow passage of sutures and metal instruments through the biologic constructs without tearing and damage. This can render the construct useless, and add significantly to the cost of the case, as these implants can be fragile as well as expensive. A damaged implant can result in several hundred dollars of added expense.

### SUMMARY

The systems and methods described below provide for delivery of sheet-like surgical implants adjacent to body tissue. The delivery device includes a frame with slotted arms, and the frame is attached to a shaft. The sheet-like implant is releasably secured to the slotted arms. The sheet-like implant is secured to the arms, and the assembled arms and implant are compressed to fit into a delivery tube, and the delivery tube is inserted into the body.

A delivery device with self-deploying sheet implants is also described. The delivery device has a slotted arm for engaging the self-deploying sheet-like implants, which are rolled onto the delivery device, withdrawn within the delivery tube, and self-deploy or open when the assembly is inserted into the surgical site extending beyond the proximal end of the delivery tube. The self-deploying sheets include spring material components made of bio-absorbable material which are attached to biologic sheet-like implants.

The self-deploying sheet may include fluid delivery capability. A top layer of the implant sheet is a biological sheet that allows dispersion of fluid through the biological sheet. The rollable frame is a grid shaped frame having a fluid inflow port at one end for introduction of fluid. The grid shaped frame includes an outer frame that contains inner members that intersect the outer frame to create internal fluid channels within the frame. Internal fluid channels allow for the flow and distribution of medications or adhesive through the channels and out of the frame via the apertures contained on the internal fluid channels.

These systems may be used for both biologic construct delivery in arthroscopy as well as other sheet and scaffold repair procedures. The system may be used for any soft tissue repair procedure where a synthetic or biologic patch is used, such as joint repair or hernia repair. A method for positioning the sheet-like surgical implant adjacent to body tissue is also described.

### BRIEF DESCRIPTION OF THE DRAWINGS

FIG. 1 illustrates a delivery device with slotted arms.

FIG. 2 illustrates the delivery device of FIG. 1 engaging a biological construct.

FIG. 3 illustrates the delivery device and biological construct of FIG. 2 collapsed and rolled for insertion.

FIG. 4 illustrates an end view of the delivery device and biological construct of FIG. 2 collapsed and rolled for insertion.

FIG. 5 illustrates a wishbone kite delivery device with straight sheet holders.

FIG. 6 illustrates a wishbone kite delivery device with curved sheet holders.

FIG. 7 illustrates a center slotted sheet holder with spring spreader arms with engagement pins.

FIG. 8 illustrates a delivery device with a single sheet holder.

FIG. 9 illustrates a delivery device with slotted holders positioned along the side of the delivery device frame.

FIG. 10 illustrates a delivery device with three sheet holders.

FIG. 11 illustrates a delivery device with a stabilizing rod.

FIG. 12 illustrates a self-deploying rolled sheet delivery device.

FIG. 13 illustrates the self-deploying rolled sheet delivery device of FIG. 12 with a self-deploying implant rolled for delivery.

FIG. 14 illustrates a top perspective view of an X-spring self-deploying sheet implant.

FIG. 15 illustrates a bottom perspective view of the X-spring self-deploying sheet implant of FIG. 14.

FIG. 16 illustrates a spring loop self-deploying sheet implant.

FIG. 17 illustrates a self-deploying sheet implant with a honeycomb spring.

FIG. 18 illustrates a self-deploying sheet implant with a barrel rib spring.

FIG. 19 illustrates the self-deploying rolled sheet delivery device of FIG. 12 engaging a quadrilateral hoop spring self-deploying sheet implant with grommet points.

FIG. 20 illustrates a self-deploying biologic sheet with a round hoop spring with molded tabs for anchor placement.

FIG. 21 illustrates an X-spring self-deploying sheet implant with grooved or slotted spring members to induce roll and flatten along one axis.

FIGS. 22A and 22B illustrate top and bottom views respectively of a self-deploying sheet implant with a serpentine spring and spike strips.

FIGS. 23A, 23B and 23C illustrate a rollable self-deploying implant sheet with fluid delivery.

### DETAILED DESCRIPTION OF THE INVENTIONS

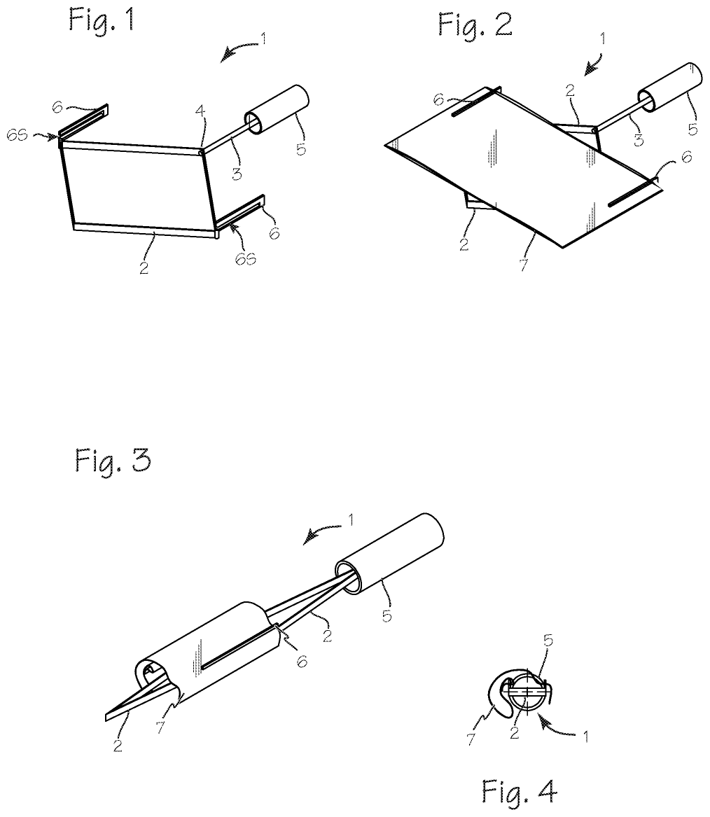

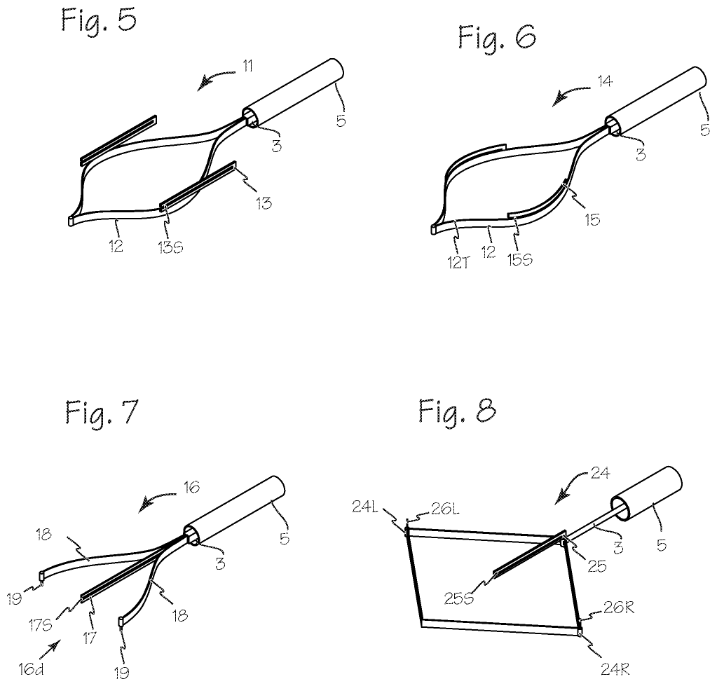

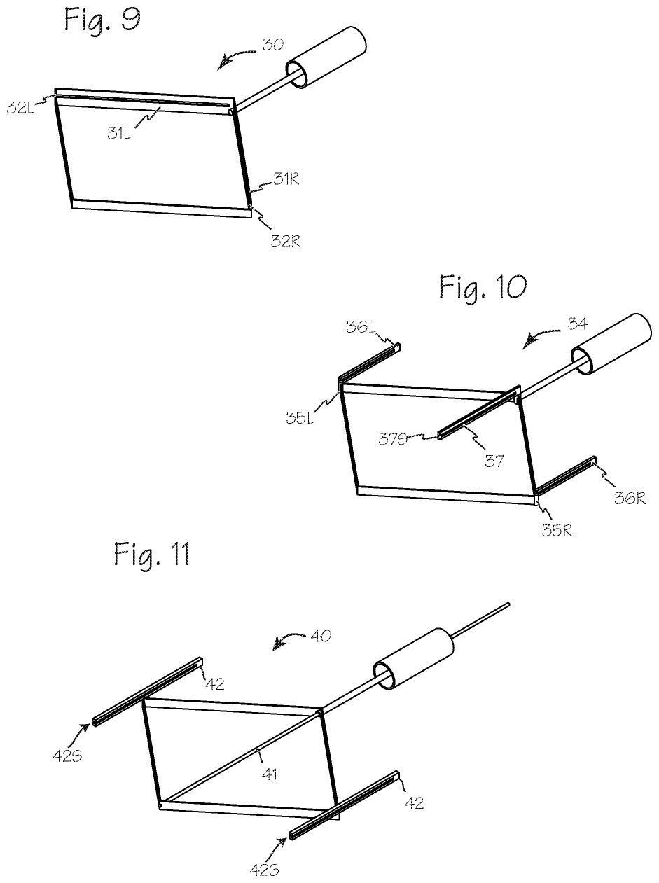

FIGS. 1 through 4 illustrate a delivery device 1 comprising a frame 2 operably connected to a shaft or handle 3. The frame as shown is a quadrilateral (rectangular, kite shaped, or other) and attached to the shaft distal end at a proximal corner of the frame 4. The shaft is slidably disposed proximally within the delivery tube 5. The frame may be made of a resilient material (spring metal, nitinol, or plastic) such that it may be compressed when pulled proximally into the delivery tube 5. The proximal corner of the frame may be hinged to facilitate compression of the frame when pulled proximally into the delivery tube.

Slotted arms 6 are disposed on side corners of the frame. The sheet-like implant or patch 7 is inserted in the slots 6S within each arm, slidably engaging the slots and thus detachably secured to the arms. The longitudinal slots run substantially the length of the arms and each slot is sized to hold a portion of the sheet-like implant by a friction fit between the arm slots 6S and the sheet-like implant 7. The slotted arms are positioned opposite each other on opposite corners of the quadrilateral frame. The arms may be fixed parallel to each other and to the shaft 3 as shown, but the arms may also be set at angles. The slot open end is on the distal end on the arms.

The frame may be quadrilateral as described, or other shapes included but not limited to forming an ellipse (FIGS. 5 and 6), circular, triangular, polyhedral or irregular-shaped frame. The frame may also be open ended, for example as shown in FIG. 7. The slotted arms are positioned on the frame parallel to the long axis of the expanded frame, or parallel to and displaced from the length of the axis of the delivery tube.

FIG. 3 illustrates the delivery device 1 collapsed and with the patch 7 rolled for insertion into the delivery tube 5. FIG. 4 illustrates an end view of the delivery device 1 collapsed with the tissue construct patch 7 rolled for insertion. The sheet-like implant is folded or rolled as shown in FIGS. 3 and 4 and compressed and loaded within the delivery tube. The sheet-like implant folds when the user retracts the shaft. Alternatively, the user may manually fold and roll the implant around the frame prior to retraction within the delivery tube.

The delivery device is retractable, and may be retracted proximally within the delivery tube, and the frame is resiliently biased toward an open configuration, as shown in FIGS. 1 and 2, and may be compressed to a closed configuration, smaller than the lumen of the delivery tube, when pulled proximally into the delivery tube, as shown in FIGS. 3 and 4. The sheet-like implant is folded or rolled as shown in FIGS. 3 and 4 and compressed and loaded within the delivery tube 5 of the delivery system 1. The frame 2 and shaft 3 are positioned within the delivery tube 5. With the components assembled, the shaft 3 is pulled proximally through the delivery tube 5 prior to delivery into a surgical space. The delivery tube may be inserted into the surgical space through a cannula or portal (not shown).

Upon full deployment distally from the delivery tube, the implant is drawn flat by the resilient expansion of the arms and frame. The sheet-like implant is positioned flat in the desired position within a surgical site and sutured or staked in place (with other instruments if necessary) or glued in place. Upon positioning and release of the implant, the frame is retracted proximally through the delivery tube and the delivery system is withdrawn from the surgical site.

FIG. 5 illustrates a delivery device 11 wherein the frame 12 is wishbone kite or ellipse shaped with straight sheet holders or arms 13. The ellipse is attached to the shaft at its proximal vertex and the arms are attached to the ellipse at the co-vertex points on the ellipse and wherein the arms 13 are arranged parallel to the shaft. The straight sheet holders or arms 13 include a slot 13S in each sheet holder, to engage the sheet-like implant. The straight sheet holders 13 are offset from the top edge, edge 12T of the delivery device. FIG. 6 illustrates a wishbone kite or ellipse delivery device 14 with curved sheet holders or arms 15 wherein the curved sheet holders follow the curve of the ellipse. The curved sheet holders 15 are offset from the top edge, edge 12T of the delivery device to create a slot 15S between the sheet holder 15 and the delivery device top edge 15T.

FIG. 7 illustrates an open-ended wishbone delivery device 16 with a center slotted sheet holder 17 with spring spreader arms 18R and 18L. The combination of the center slotted sheet holder with the spring spreader arms creates a fork-like delivery device with an open distal end 16d. The distal ends of the spring spreader arms 18R and 18L are fitted with engagement pins 19 for securing and spreading a biological construct sheet engaged in the slot 17S of the center slotted sheet holder 17. The spreader arms are made of a resilient material (spring metal, nitinol, or plastic) such that they spring open when deployed distally from the distal end of the delivery tube. A first deployment arm and a second deployment arm each have a proximal and a distal end with the proximal end of each arm coupled to the distal end of the shaft. The first and second deployment arms are moveable between a closed position and an open position wherein in the closed position the arms extend generally in the longitudinal direction and in pivoting to the open position the distal end of each arm moves in a generally transverse direction to spread the sheet-like implant. The distal end of the arms are biased inward or toward each other as shown to form the wishbone shape. The proximal segment of each arm bends toward the center of the tube to join the shaft at their proximal ends. The shaft is disposed in a lumen of the delivery tube, and longitudinally translatable within the lumen, extendable distally to the sheet-like implant and operable to hold the implant in place.

FIG. 8 illustrates a delivery device 24 with a single sheet holder 25. The lateral vertices 24L and 24R of the delivery device are equipped with engagement pins 26L and 26R respectively, for securing and spreading a biological construct sheet engaged in slot 25S of the center slotted sheet holder 25.

FIG. 9 illustrates a delivery device 30 with the proximal arms 31L and 31R having slots 32L and 32R respectively. The proximal arms are along the edges of the rectangular frame that are adjacent sides of the rectangle joined at the rectangle proximal corner. The engagement slots 32L and 32R engage the sheet-like implant.

FIG. 10 illustrates a delivery device 34 with three slotted sheet holders. The lateral vertices 35L and 35R of the delivery device are equipped with slotted engagement arms 36L and 36R respectively for securing and spreading a biological construct sheet engaged in slot 37S of the center sheet holder 37. The side engagement arms are attached to face in the proximal direction and the center sheet holder extends in the distal direction. All slots are open at the distal end of their respective arms.

FIG. 11 illustrates a delivery device 40 with a stabilizing rod 41 extending from the proximate corner to the distal corner of the rectangular delivery device. Slotted sheet holders 42 extend across the frame side corners with a portion of the arm extending proximally and distally such that the approximate midpoint of the arms are attached to the rectangle opposing side corners.

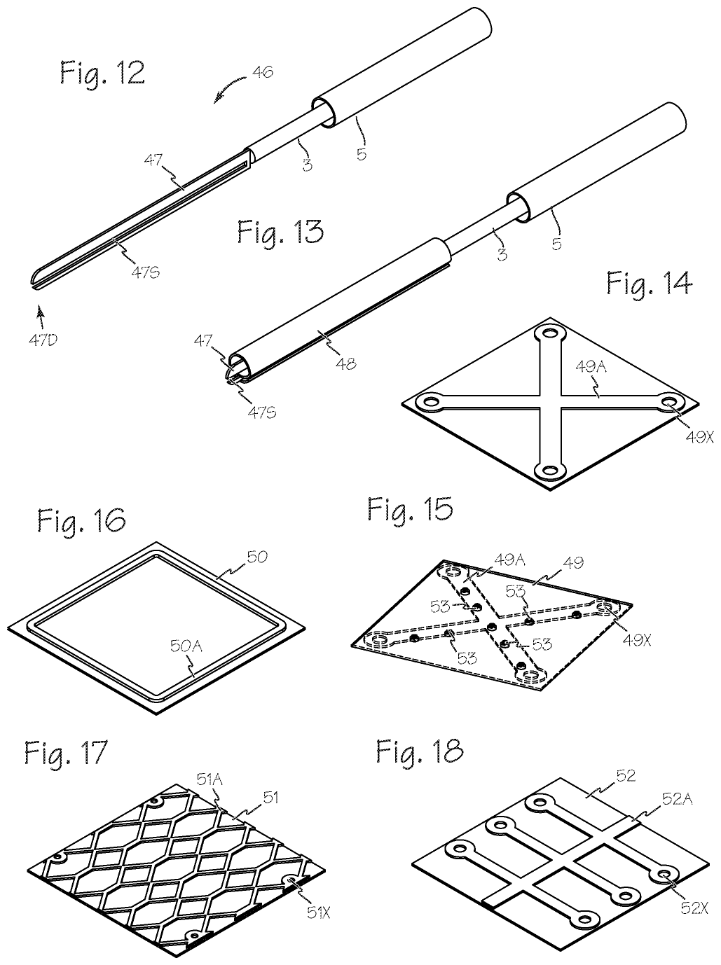

FIG. 12 illustrates a delivery device 46 for a self-deploying rolled sheet implant. The delivery device includes an engagement arm 47 which has a single slot 47S with the slot open end at the distal end 47D of the arm. The delivery device engages, within the slot, the self-deploying implants described below in FIGS. 14 through 22b. FIG. 13 illustrates the self-deploying rolled sheet delivery device 46 with a self-deploying implant 48 rolled upon the engagement arm 47 for delivery. The self-deploying implants are engaged within the delivery device slot in their small diameter configuration, rolled onto the delivery device, and self-deploy when placed within the surgical site in their open flat configuration. The device with the self-deploying implants is retractable within the insertion tube 5.

FIGS. 14 through 18 illustrate alternate self-deploying sheet implants for use with the delivery device 46 shown in FIG. 12. The implants are resiliently biased toward an open flat configuration. Self-deploying sheets such as sheets 49, 50, 51 and 52 include spring material components 49A, 50A, 51A and 52A. Present as a preferred option, the spring material components are made of laser or die cut Poly (D,L-lactic acid) (PLA) and/or poly (D,L-lactic-co-glycolic acid)(PLGA) biodegradable polymers or other suitable bio-absorbable material or biocompatible metal. The spring material components 49A, 50A, 51A and 52A are attached to their respective biologic sheets by sutures, staking, adhesives or laminating the spring material components between biologic implant sheets. For example, the “X-spring” spring material component 49A (FIG. 14) is secured to biologic sheet 49 by staked posts 53 as shown on the underside view (FIG. 15). The spring material components 49A, 50A, 51A and 52A may also include grommet points such as grommet points 49X, 51X and 52X to engage tissue anchors. Spring loop 50A (FIG. 16) is configured as a quadrilateral shape with a 45° angle to reflect the open configuration of the “kite” deployment devices disclosed above. Any other suitable shapes may be used for the spring loop such as a circle, oval, rectangular or other complex shape. The stent sheet spring 51A (FIG. 17) may provide more uniform support for a fragile biologic sheet and comprises a webbing or honeycomb pattern. Barrel rib spring 52A (FIG. 18) is another alternate configuration for a self-deploying spring material component wherein ribs extend outwardly perpendicular to the center strip.

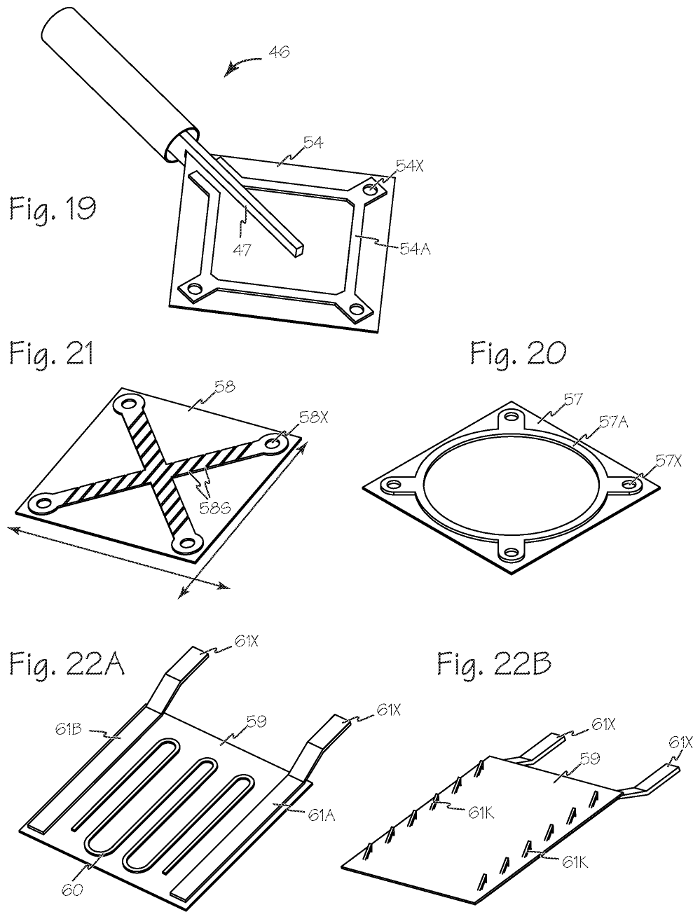

FIG. 19 illustrates the self-deploying rolled sheet delivery device of FIG. 12 engaging a quadrilateral self-deploying sheet implant 54 with hoop spring 54A incorporating molded tabs 54X for anchor placement.

FIG. 20 illustrates a self-deploying biologic sheet 57 with a round hoop spring 57A with molded tabs 57X for anchor placement. Hoop springs such as springs 50A, 54A and 57A may adopt any suitable shape such as rectangular, rhomboid, round, ellipsoid or any other simple or complex shape.

FIG. 21 illustrates a self-deploying sheet implant 58 with an “X-spring” spring material component 58A. One or more slots, grooves or other indentations such as slots 58S may be formed or cut into one or both surfaces of the spring member to permit the spring member to roll parallel to one axis and flatten along another orthogonal axis.

FIGS. 22A and 22B illustrate top and bottom views respectively of a self-deploying sheet implant 59 with a serpentine shaped spring 60 on first side 59A and tabbed spike strips 61A and 61B on second, opposite or obverse side 59B. Each spike strip, strips 61A and 61B, contain any suitable number of staple spikes such as spikes 61K. The spike strips 61A and 61B are pressed down with a tool, and the tabs 61X are pulled to set the respective staple spikes into tendon. Tabs 61X are clipped off after the spike strip is secured to the tissue in the surgical site. The spike strip and “S” spring may be molded of any suitable bioabsorbable polymer.

In use, the surgeon delivers the implant to a joint within the body of a patient by creating an arthroscopic workspace around the joint and inserting a cannula through the skin of the patient proximate the arthroscopic workspace. The surgeon attaches the implant to the delivery device and retracts the shaft with the sheet-like implant attached such that the delivery device and sheet fit through the delivery tube within the cannula. The surgeon inserts the delivery tube with delivery device with attached sheet-like implant assembly through the cannula and into the arthroscopic workspace. The surgeon pushes the shaft in a distal direction to extend the delivery device with implant within the workspace and positioning the sheet proximate an intended site of implantation.

The surgeon then secures the sheet to body tissue within the workspace. The implant is secured with staples, sutures, clips or other means, with a separate instrument. The implant may also be secured with a tissue adhesive. Once the implant is essentially staked in place, the surgeon releases the implant from the delivery device by sliding the delivery device proximally, whereupon the implant is slidably released from the delivery device arms when the delivery device is withdrawn proximally into the delivery tube. The surgeon retracts the shaft within the delivery tube and removes the implant delivery system from the workspace.

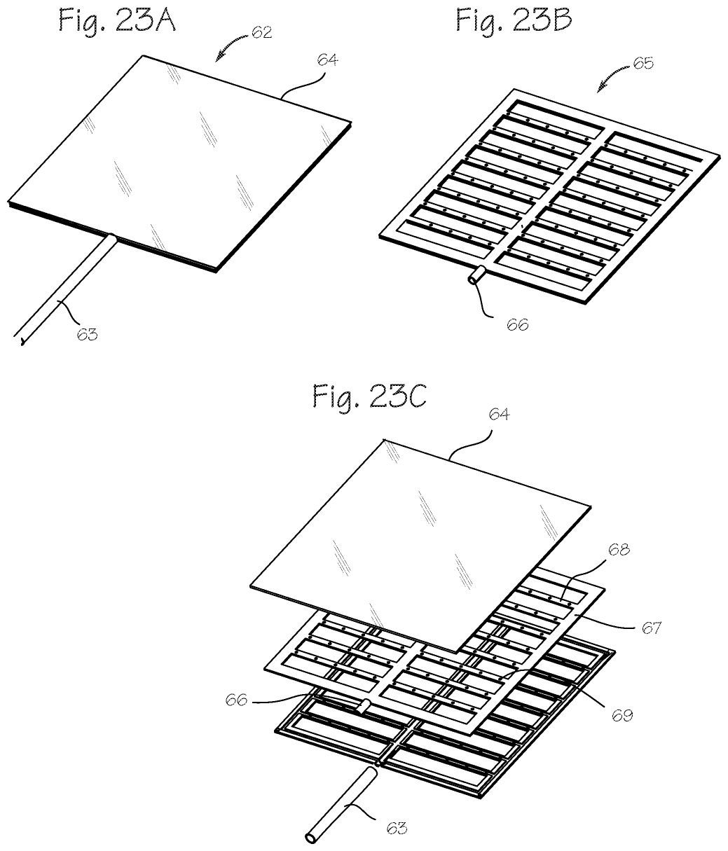

FIGS. 23A, 23B and 23C illustrate a rollable self-deploying implant sheet with fluid delivery. FIG. 23A is an over-all view of the self-deploying sheet implant 62 attached to a detachable fluid delivery tube 63. The top layer of the implant sheet is a biological sheet 64 that allows dispersion of fluid through the biological sheet. The self-deploying sheet implant 62 includes a rollable frame 65 with a fluid inflow port 66. FIG. 23C is an exploded view of rollable self-deploying implant sheet 62. The top layer is the biological sheet 64. The rollable frame 65 is shown in an exploded view 65T and 65B. The rollable frame 65 is a grid shaped frame having the fluid inflow port 66 at one end for introduction of fluid. The grid shaped frame includes an outer frame 67 that contains inner members 68 that intersect the outer frame to create internal fluid channels within the frame. The internal fluid channels allow for the flow and distribution of medications or adhesive through the channels and out of the frame via the apertures 69 contained on the internal fluid channels.

The frame may be a micromolded grid shaped frame made of any rollable thermoplastic and bio-reabsorbable material. The frame may be a unitary piece or a multi component frame that has separate molded components ultrasonically or laser welded together. The internal fluid channels can be made of any heat-sealed bio-absorbable polymer film or open cell foam material. In addition, the polymer film may be perforated with laser-drilled holes. The frame provides structural support for cells residing in tissue to attach, grow and migrate. The implant provides a degradable physical environment to allow neovascularization and remodeling in response to developmental, physiological and pathological challenges during tissue dynamic processes and wound healing. The implant can be delivered to the target tissue via any of the delivery devices disclosed herein.

In use, fluid is introduced into the fluid inflow port via the detachable delivery tube 63. The fluid is conducted through the internal fluid channels of the grid shaped frame and out of the apertures on the vertical sides. The fluid flows from the apertures, through the biological sheet for contact with human tissue. The fluid medications or adhesives are then locally delivered to the interface between the biologic sheet and target tissue on the patient. Medications or adhesives may include stem cell or platelet rich plasma (PRP) locally between the biologic sheet and the target tissue. This can be useful for bio-inductive implants or constructs, in a fluid mediated arthroscopic procedure, or useful in a gas-mediated arthroscopic or endoscopy procedure where the biological implant or construct is fixed to the target tissue with an adhesive such as fibrin glue. The implant can be used for tendon or cartilage repair and can be attached with staples or anchors or fixed in place with locally delivered adhesives.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

1.-23. (canceled)

24. A rollable sheet implant comprising:
a top layer comprising a biological sheet; and
a bottom layer comprising a spring material biased to an open flat configuration, said bottom layer further comprising a bio-absorbable material.

25. The rollable sheet implant of claim 24 wherein the bottom layer forms an “x” pattern.

26. The rollable sheet implant of claim 24 wherein the bottom layer is a honeycomb pattern.

27. The rollable sheet implant of claim 24 wherein the bottom layer has a center strip with a plurality of ribs extending outwardly from and perpendicular to the center strip.

28. The rollable sheet implant of claim 24 wherein the bottom layer is a quadrilateral with molded tabs disposed on the corners of the quadrilateral.

29. The rollable sheet implant of claim 24 wherein the bottom layer is a round shaped hoop with a plurality of molded tabs ^{d}isposed on the hoop.

30. The rollable sheet implant of claim 24 wherein the bottom layer is a serpentine shaped spring disposed on a first side of the bottom layer and a plurality of tabbed spike strips on a second side of the bottom layer, wherein the first side of the bottom layer is in contact with the top layer.

31. The self-deploying rollable sheet implant of claim 24 wherein:
the biological sheet allows dispersion of fluid through the biological sheet;
the bottom layer further comprises a frame having an outer frame and inner fluid channels within the outer frame;
wherein the bottom layer includes a fluid inflow port at a first end for introduction of fluid; and
wherein the inner fluid channels include apertures that allow for disbursement of fluid through the apertures.

32-39. (canceled)
